(19) United States
(12) Patent Application Publication
(10) Pub. No.: US 2008/0188780 A1
(43) Pub. Date: Aug. 7, 2008

(54) SPINAL DISTRACTION DEVICE WITH THREE DIMENSIONALLY VIBRATING MATRIX HEAD

(75) Inventor: Carlos Becerra, Atlanta, GA (US)

Correspondence Address: BRYAN W. BOCKHOP, ESQ.;BOCKHOP & ASSOCIATES, LLC, 2375 MOSSY BRANCH DR., SNELLVILLE, GA, 30078, US

(73) Assignee: NORTH AMERICAN MEDICAL CORPORATION, Marietta, GA (US)

(21) Appl. No.: 12/023,106

(22) Filed: Jan. 31, 2008

Related U.S. Application Data
(60) Provisional application No. 60/887,953, filed on Feb. 2, 2007.

(51) Int. Cl. A61H 1/00; A61F 5/00

(52) U.S. Cl. 601/49; 602/33; 602/36; 601/33; 601/46

(57) ABSTRACT

A spinal treatment system for applying a preselected force regimen to a spine of a patient includes a treatment bed, having a head end and a foot end, is configured to support the patient. A harness is configured to transmit force to a portion of the spine of the patient. A cable having a first end is coupled to the harness and is configured to apply force to the harness. A matrix head is coupled to the cable and is configured to apply a force, having a force magnitude and a force direction, to the cable, so that the force direction varies along more than one axis. A vertical displacement drive is disposed adjacent to the foot end of the treatment bed and is configured to support the matrix head and to displace the matrix head along a vertical axis. A computer is programmed to drive the vertical displacement drive and the matrix head.

### CROSS-REFERENCE TO RELATED APPLICATION(S)

This application claims the benefit of U.S. Provisional Patent Application Ser. No. 60/887,953, filed Feb. 2, 2007, the entirety of which is hereby incorporated herein by reference.

### BACKGROUND OF THE INVENTION

1. Field of the Invention

The present invention relates to medical devices and, more specifically, to a device for applying forces to a patients spine.

2. Description of the Prior Art

Spinal distraction devices treat lower back pain by applying a decompression force to selected vertebrae of a patient. Existing spinal distraction devices apply either a static force on the patent or a simple oscillating force to the patient. The use of oscillations allows the patient to relax while the force is being applied, thereby facilitating the treatment. The simple oscillation patterns, however, tend to relax only certain muscles in the spinal area and, thus, fail to achieve complete relaxation quickly. Complete relaxation makes spinal decompression more effective.

Therefore, there is a need for spinal distraction that achieves more complete relaxation.

### SUMMARY OF THE INVENTION

The disadvantages of the prior art are overcome by the present invention which, in one aspect, is a spinal treatment system for applying a preselected force regimen to a spine of a patient. A treatment bed, having a head end and a foot end, is configured to support the patient. A harness is configured to transmit force to a portion of the spine of the patient. A cable having a first end is coupled to the harness and is configured to apply force to the harness. A matrix head is coupled to the cable and is configured to apply a force, having a force magnitude and a force direction, to the cable, so that the force direction varies along more than one axis. A vertical displacement drive is disposed adjacent to the foot end of the treatment bed and is configured to support the matrix head and to displace the matrix head along a vertical axis. A computer is programmed to drive the vertical displacement drive and the matrix head.

In another aspect, the invention is a matrix head for use in a spinal treatment system. A cable tensioning unit is configured to apply a selected tension to a cable. A vertical vibration unit is configured to apply vertical vibrations to the cable tensioning unit. A horizontal vibration unit is configured to apply horizontal vibrations to the cable tensioning unit.

In yet another aspect, the invention is a method of performing spinal distraction on a patient, in which a preselected tensile force is applied to a selected spinal location of the patient for a preselected period. The selected spinal location is vibrated according to a preselected vibrational pattern during the preselected period.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is an elevational view of a spinal decompression structure according to one embodiment of the invention.

FIG. 2 is a cross-sectional view of a three dimensional vibration matrix head.

FIG. 3 is a cross-sectional view of a portion of a patient harness.

FIGS. 4A-4E are diagrams of vibration profiles.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

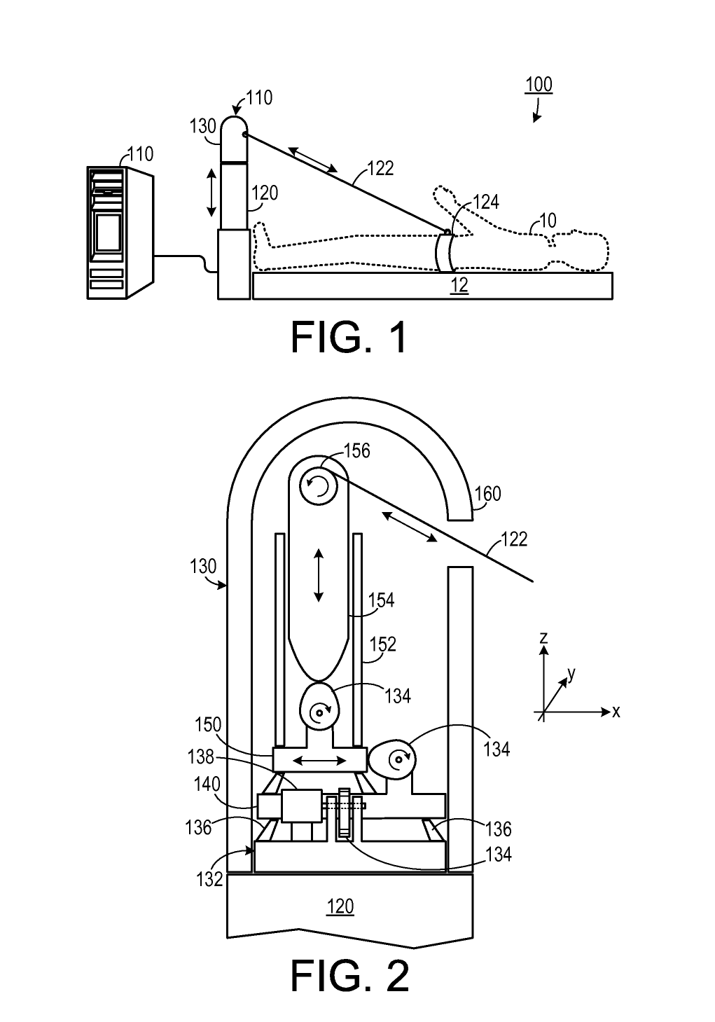

As shown in FIG. 1, one embodiment of the invention is a spinal treatment system 100 that includes a treatment bed 12 upon which a patient 10 reclines and a spinal distraction device 110 that includes a mechanism 120 for adjusting a vertical displacement of a matrix head 130, thereby adjusting the angle at which force is applied to the patient 10 through a cable 122 (such as a steel cable). The cable 122 applies force to a harness 124 that transfers the force to the patient 10. The matrix head 130 is controlled by a computer 110, which controls the matrix head 130 to cause it to apply a selected vibrational profile to the cable 122, while applying a preselected tension to the cable 122 at a preselected angle to the patient 10.

As shown in FIG. 2, the matrix head 130 includes three vibration units that each apply vibration along a different axis. A fore-aft vibration unit 132, which applies vibration along the y-axis, includes a motor 138 that drives a cam 134 and a guide 136 upon which rests a left-right vibration unit 140. The motor 138 causes the cam 134 to rotate, which imparts vibrational force on the left-right vibration unit 140. The left-right vibration unit 140, which applies vibration along the x-axis, includes a motor that drives a cam 134 and a guide 136 upon which rests an up-down vibration unit 150. Rotation of the motor-driven cam 134 of the left-right vibration unit 140 imparts vibrational force on the up-down vibration unit 150. The up-down vibration unit 150 applies vibrational force along the z-axis to a tensioning unit 156. The up-down vibration unit 150 includes a motor-driven cam 134 that applies vibrational force to a piston 154 that is guided by a cylinder 152. The tensioning unit 156 is coupled to the piston 154 and applies a predetermined amount of tension to the cable 122. A protective cover 160 may be used to cover the matrix head 130.

While cam-based vibration units are shown in the figures, it should be understood that many other types of vibrators may be used in the invention. For example, solenoid-type vibrators and acoustic transducers may also be used.

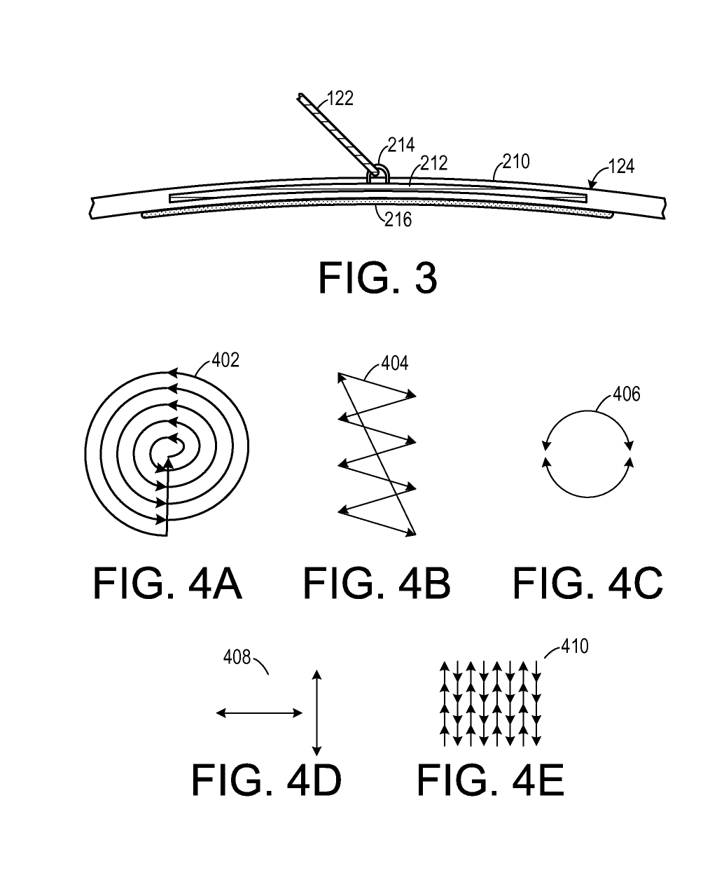

The harness should be rigid enough to transfer vibrations to the patient over an area sufficient for efficacy of the treatment. One illustrative embodiment of the harness 124, as shown in FIG. 3, includes a rigid member 212 that is coupled to a loop 214. The loop 214 is coupled to the cable 122. The rigid member 212 may be constructed of such materials as a plastic, a metal, a composite or other rigid materials. The rigid member 212 may also be enveloped by a collar 210, which could be made of a material that is compliant that the rigid member 212, to act as an interface to the patient. An adhesive 216 may be used to secure a portion of the harness 124 to the patient, thereby facilitating the transfer of vibrational energy to the patient. Preferably, the adhesive 216 would be a hypo-allergenic adhesive.

The motors driving the cams 134 may be controlled by a computer 110 so that the matrix head 130 vibrates according to a predefined pattern. Examples of such patterns are shown in FIGS. 4A-4E and includes: a repeating spiral 402, a zig-zag 404 (which may be oriented in any manner indicated), half circles 406, alternating up-down/left-right 408, and simple direction reversing patterns 410. It is understood that FIGS. 4A-4E disclose only a partial set of vibrational patterns that may be used with the invention and that many other types of patterns could be used as part of the invention.

Choice of the type of cable 122 used can depend on the frequency and vibrational pattern being used. For example, a cable made of a more rigid material might be used when higher frequency vibrations are being applied, whereas a cable made of a less rigid material might be used with lower frequency vibrations. Also, the width of the cable 122 might also be associated with frequency and vibrational pattern. In one embodiment, the cable 122 is a steel cable.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A spinal treatment system for applying a preselected force regimen to a spine of a patient, comprising:
a. a treatment bed, having a head end and a foot end, configured to support the patient;
b. a harness configured to transmit force to a portion of the spine of the patient;
c. a cable having a first end coupled to the harness and configured to apply force to the harness;
d. a matrix head that is coupled to the cable and that is configured to apply a force, having a force magnitude and a force direction, to the cable, so that the force direction varies along more than one axis;
e. a vertical displacement drive disposed adjacent to the foot end of the treatment bed configured to support the matrix head and to displace the matrix head along a vertical axis; and
f. a computer that is programmed to drive the vertical displacement drive and the matrix head.

2. The spinal treatment system of claim 1, wherein the matrix head comprises:
a. a cable tensioning unit configured to apply a selected tension to the cable;
b. a vertical vibration unit configured to apply vertical vibrations to the cable tensioning unit; and
c. a horizontal vibration unit configured to apply horizontal vibrations to the cable tensioning unit.

3. The spinal treatment system of claim 2, wherein the horizontal vibration unit comprises a first horizontal vibration member configured to apply horizontal vibrations along a first horizontal axis.

4. The spinal treatment system of claim 3, wherein the horizontal vibration unit comprises a second horizontal vibration member configured to apply horizontal vibrations along a second horizontal axis that is transverse to the first horizontal axis.

5. The spinal treatment system of claim 1, wherein the computer is programmed to drive the vertical displacement drive and the matrix head so that the cable imparts a preselected vibrational pattern onto the cable.

6. The spinal treatment system of claim 1, wherein the harness comprises:
a. a harness portion configured to engage the patient at a selected location;
b. a rigid member, affixed to a top portion of the harness portion, that is configured to transmit forces applied thereto to the harness portion; and
c. a connection member that is configured to couple the first end of the cable to the harness, the connection member mechanically coupled to the rigid member.

7. A matrix head for use in a spinal treatment system, comprising:
a. a cable tensioning unit configured to apply a selected tension to a cable;
b. a vertical vibration unit configured to apply vertical vibrations to the cable tensioning unit; and
c. a horizontal vibration unit configured to apply horizontal vibrations to the cable tensioning unit.

8. The matrix head of claim 7, wherein the horizontal vibration unit comprises a first horizontal vibration member configured to apply horizontal vibrations along a first horizontal axis.

9. The matrix head of claim 8, wherein the horizontal vibration unit comprises a second horizontal vibration member configured to apply horizontal vibrations along a second horizontal axis that is transverse to the first horizontal axis.

10. The matrix head of claim 7, further comprising a controller that is configured to control the vertical vibration unit and the horizontal vibration unit.

11. The matrix head of claim 10, wherein the controller comprises a processor that is programmed to generate a control signal that causes both the vertical vibration unit and the horizontal vibration unit to vibrate according to a predetermined pattern of vibrations.

12. The matrix head of claim 7, further comprising a vertical displacement adjustment mechanism disposed so as to support the matrix head and that is configured to adjust a vertical displacement of the matrix head.

13. A method of performing spinal distraction on a patient, comprising the actions of:
a. applying a preselected tensile force to a selected spinal location of the patient for a preselected period; and
b. vibrating the selected spinal location according to a preselected vibrational pattern during the preselected period.

14. The method of claim 13, wherein the action of applying a preselected tensile force comprises the actions of:
a. securing a harness to the patient at the selected spinal location;
b. coupling the harness to a cable; and
c. controlling tension on the cable using a matrix head.

15. The method of claim 14, wherein the vibrating action comprises applying selected vibrations to the matrix head so that the matrix head imparts the preselected vibrational pattern onto the cable.

16. The method of claim 14, wherein the, controlling action comprises generating a control signal with a digital processor, thereby activating the matrix head.

17. The method of claim 13, wherein the preselected vibrational pattern follows a repetitive spiral path.

18. The method of claim 13, wherein the preselected vibrational pattern follows a repetitive zigzag path.

19. The method of claim 13, wherein the preselected vibrational pattern follows a repetitive curved path.

20. The method of claim 13, wherein the preselected vibrational pattern follows a repetitive alternating horizontal and vertical path.
